(19) United States
(12) Patent Application Publication
(10) Pub. No.: US 2007/0009479 A1
(43) Pub. Date: Jan. 11, 2007

(54) Methods for treating dermatitis using mutant human IL-4 compositions

(75) Inventor: Richard Fuller, Walnut Creek, CA (US)

Correspondence Address: DLA PIPER US LLP, 4365 EXECUTIVE DRIVE, SUITE 1100, SAN DIEGO, CA, 92121-2133, US

(73) Assignee: Aerovance, Inc., Berkeley, CA (US)

(21) Appl. No.: 11/156,221

(22) Filed: Jun. 17, 2005

(51) Int. Cl. A61K 38/20; C07K 14/54; C08G 63/48

(52) U.S. Cl. 424/85.2; 525/54.1; 530/351

(57) ABSTRACT

Methods of administering a therapeutically effective amount of a mutant human IL-4 composition to a human subject for the amelioration and treatment of dermatitis, including contact and atopic dermatitis.

### BACKGROUND INFORMATION

1. Field of the Invention

The present invention relates to methods for treating atopic diseases, including atopic dermatitis and other inflammatory or allergic skin disorders by administering mutant human Interleukin-4 (IL-4) compositions that act as antagonists to IL-4 and IL-13.

2. Background of the Invention

Interleukin-4 (IL-4) is a pleiotropic cytokine with a broad spectrum of biological effects on several target cells, including activation, proliferation and differentiation of T and B cells. IL-4 is increasingly appreciated as a pivotal cytokine initiating the “Th2-type” inflammatory response whereas IL-13 is now appreciated as the more probable downstream effector cytokine. During proliferation of B-lymphocytes, IL4 acts as a differentiation factor by regulating class switching to the IgG1 and IgE isotypes.

Atopic diseases are characterized by formation of IgE antibodies, which results in immediate hypersensitivity reactions upon exposure to specific allergens. The frequent and chronic infections occurring on the skin of atopic disease patients results from the impaired immune response and from the skin barrier breaking down. Known treatments of atopic diseases include, hydrating the skin, dietary restrictions, avoidance of irritants and allergens in the environment, tars, antihistamines, hyposensitization, corticosteroids, antibacterials, antifungals, ultraviolet light, leukotriene blockers, inhibitors of mast cell content release, pentoxifylline, azathioprine, cyclosporin A, cyclophosphamide, tacrolimus, interferon gamma, thymopentin and phosphodiesterase inhibitors.

Generally, anti-histamine and steroidal agents are used as therapeutic treatments for atopic diseases. Anti-histamine agents typically reduce the itchiness of the allergic response and include diphenhydramine hydrochloride, mequitazine, promethazine hydrochloride, and chlorpheniramine maleate. Steroidal agents including prednisolone, hydrocortisone butyrate, dexamethasone valerate, betamethasone dipropionate, clobetasol propionate and the like have also been used to control the itching. While anti-histamine and steroidal agents relieve the itching, they are not desireable therapeutic agents because they cause other adverse side affects including infection, secondary adrenal cortical insufficiency, diabetes, peptic ulcer, hirsutism, alopecia, and pigmentation.

### BRIEF DESCRIPTION OF THE DRAWINGS

FIG. 1 is a graph showing wheal response measurements without clinical score after vehicle control (PBS) treatment.

FIG. 2 is a graph showing wheal response measurements with clinical score after vehicle control (PBS) treatment.

FIG. 3 is a graph showing wheal response measurements without clinical score after mutant human IL-4 (mhIL-4) treatment (1 mg/kg, sid s.c. 3×/week).

FIG. 4 is a graph showing wheal response measurements with clinical score after mhIL-4 treatment (1 mg/kg, sid s.c. 3×/week).

FIG. 5 is a graph showing wheal response measurements and plasma levels of IgE after mhIL-4 treatment (1 mg/kg, sid s.c. 3×/week).

FIG. 6 is a graph showing the change in IL-4 binding to IL-4 receptor alpha in surface plasmon resonance units after mhIL-4 treatment (1 mg/kg, sid s.c. 3×/week).

### SUMMARY OF THE INVENTION

The present invention provides methods for suppressing or inhibiting a dermatitis response in a subject, including a method of treating atopic diseases by administering a mutant human IL-4 (mhIL-4) therapeutic composition.

In one embodiment of the invention, there is provided a method for suppressing or inhibiting a dermatitis response in a subject in need thereof a therapeutic effective amount of a mutant human IL-4 protein, or functional fragment thereof, wherein the protein or fragment thereof comprises at least a first modification of replacing one or more of the amino acids occurring in the wild-type human IL-4 protein at positions 121, 124 and/or 125 with another natural amino acid.

In another embodiment of the invention, there is provided a method for treating a subject having atopic dermatitis, including administering to the subject having atopic dermatitis a therapeutically effective amount of a composition comprising a human IL-4 protein, wherein the mutein comprises a first modification of replacing one or more of the amino acids occurring in the wild-type human IL-4 protein at positions 121, 124 and/or 125 with another natural amino acid; and a second modification selected from a group consisting of a modification at the N-terminus, C-terminus, deletion of potential glycosylation sites therein, and/or coupling of the protein to a non-protein polymer, and wherein the therapeutic effective amount is from about 0.3 mg/kg to about 0.6 mg/kg daily.

### DETAILED DESCRIPTION

The invention provides for methods of treatment of atopic diseases (AD), in particular, atopic dermatitis, by administering a therapeutic effective amount of mutant human IL-4 compositions. Human IL-4 mutant proteins used as antagonists or partial agonists of human IL-4 are also described in U.S. Pat. No. 6,130,318 to Wild et al., the entire contents of which is incorporated herein by reference.

The methods of the invention can be used to treat typical atopic diseases or allergic dermatitis including contact dermatitis, atopic dermatitis (i.e., eczema), psoriasis, seborrheic dermatitis, and the like. As used herein, the term “dermatitis” is defined generally as an inflammation of the skin. Stedman's Medical Dictionary, 27th edition, Lippincott Williams & Wilkins (2000).

As used herein, the term “contact dermatitis” is an inflammatory response of the skin to an antigen (or allergen) or irritant (Stedman's Medical Dictionary, supra). Irritants are substances that directly affect the skin or cause direct tissue damage, while allergens induce an immunologic reaction that causes inflammation and tissue damage. Some common irritants are wool and synthetic fibers, soaps and detergents, perfumes and cosmetics, dust and sand, cigarette smoke, and substances such as chlorine, mineral oil or solvents.

Allergens are substances typically from foods, plants, or animals that inflame the skin and cause an immune reaction. Initially, allergens typically illicit inflammatory response, including recruitment of cells, for example T cells, macrophages and the like. Upon repeated contact with the allergen, the contact dermatitis then develops into eczema accompanied with lichenification and infiltration of the cells.

As used herein, the term “atopic dermatitis,” “atopic eczema,” or “eczema” and related terms are used interchangeably and represent a complex disease primarily caused by cellular immune deficiency and elevated immunoglobulin E (IgE). Allergens that are also irritants to the skin are believed to predispose an individual to develop dermatitis more often than simply exposure to an allergenic trigger. Anxiety, stress and depression may all play a role in the exacerbation of eczema. Further, those with atopic eczema may be discovered to have an increased eosinophil count.

As used herein, the terms “mutant human IL-4 protein,” “modified human IL-4 receptor antagonist,” “mhIL-4,” “IL-4 antagonist,” and equivalents thereof are used interchangeably and are within the scope of the invention. These polypeptides and functional fragments thereof refer to polypeptides wherein specific amino acid substitutions to the mature human IL-4 protein have been made. These polypeptides include the mIL-4 compositions of the present invention, which are administered to a subject in need of treatment thereof. In particular, the mhIL-4 of the present invention, include at least the R121D/Y124D pair of substitutions. Other embodiments of the mhIL-4 polypeptides are discussed herein. As used herein, a “functional fragment” is a polypeptide which has IL-4 antagonistic activity, including smaller peptides. These and other aspects of mhIL-4 of modification of hIL-4 are described in U.S. Pat. Nos. 6,335,426; 6,313,272; and 6,028,176, the entire contents of which are incorporated herein by reference.

As used herein, “wild type IL-4” or “wtIL-4” and equivalents thereof are used interchangeably and mean human Interleukin-4, native or recombinant, having the 129 normally occurring amino acid sequence of native human IL-4, as disclosed in U.S. Pat. No. 5,017,691, incorporated herein by reference. Further, the modified human IL-4 receptor antagonists described herein have various insertions and/or deletions and/or couplings to a non-protein polymer are numbered in accordance with the wtIL-4, which means that the particular amino acid chosen is that same amino acid which normally occurs in the wtIL-4. In one embodiment, one skilled in the art will appreciate that, the normally occurring amino acids at positions, for example, 121 (arginine), 124 (tyrosine), and/or 125 (serine), may be shifted in the mutein. In another embodiment, one skilled in the art will appreciate that an insertion of a cysteine residue to amino acid positions, for example, 38, 102 and/or 104 may be shifted on the mutein. However, the location of the shifted Ser (S), Arg (R), Tyr (Y) or inserted Cys (C) can be determined by inspection and correlation of the flanking amino acids with those flanking Ser, Arg, Tyr or Cys in wtIL-4.

In one embodiment of the invention, a modified human IL-4 receptor antagonist is useful for treating various conditions associated with one of the pleiotropic effects of IL-4 and IL-13. For instance, antagonists of IL-4 and IL-13 are useful in treating conditions exacerbated by IL-4 and IL-13 production including asthma, allergy, or other inflammatory response-related conditions. Some uses of the modified human IL-4 mutein receptor antagonists are described in U.S. Pat. No. 6,130,318, the entire contents of which is incorporated herein by reference. MHIL-4, a therapeutic compound of the invention, is a form of a modified human IL-4 mutein receptor antagonist as described in U.S. Pat. No. 6,130,318.

Antagonists to IL-4 have been reported in the literature. Mutants of IL-4 that function as antagonists include the IL-4 antagonist mutein IL-4/Y124D (Kruse, N., Tony, H. P., Sebald, W., Conversion of human interleukin-4 into a high affinity antagonist by a single amino acid replacement, Embo J. 11:3237-44, 1992) and a double mutein IL-4[R121D/Y124D] (Tony, H., et al., Design of Human Interleukin-4 Antagonists in Inhibiting Interleukin-4-dependent and Interleukin-13-dependent responses in T-cells and B-cells with high efficiency, Eur. J. Biochem. 225:659-664 (1994)). The single mutein is a substitution of tyrosine by aspartic acid at position 124 in the D-helix. The double mutein is a substitution of Arginine by Aspartic Acid at position 121, and of tyrosine by aspartic acid at position 124 in the D-helix. Variations in this section of the D helix positively correlate with changes in interactions at the second binding region.

In another embodiment of the invention, the modified IL-4 receptor antagonist polypeptide, or a functional fragment thereof, is coupled to a non-protein polymer at various amino acid residues, in particular, at positions 28, 36, 37, 38, 102, 104, 105 or 106. The amino acid positions are numbered according to the wild type IL-4 (i.e. human interleukin-4) amino acid sequence (see U.S. Pat. No. 5,017,691 which is incorporated herein by reference). In another aspect of the invention, the amino acid residue at positions 28, 36, 37, 38, 104, 105 or 106 is cysteine. These and other aspects of covalent modification of mutant human IL-4 to non-protein polymer are described in U.S. application Ser. No. 10/820,559, filed Apr. 8, 2004, U.S. Pat. Nos. 4,640,835; 4,496,689; 4,301,144; 4,670,417; 4,791,192 or 4,179,337, the entire contents of which are incorporated herein by reference.

A skilled artisan will be able to determine suitable variants of the polypeptide and functional fragment thereof as set forth herein using well-known techniques. In certain embodiments, one skilled in the art may identify suitable areas of the polypeptide that may be changed without destroying activity by targeting regions not believed to be important for activity. In other embodiments, the skilled artisan can identify residues and portions of the polypeptides that are conserved among similar polypeptides. In further embodiments, even areas that may be important for biological activity or for structure may be subject to conservative amino acid substitutions without destroying the biological activity or without adversely affecting the polypeptide structure.

Additionally, one skilled in the art can review structure-function studies identifying residues in similar polypeptides that are important for activity or structure. In view of such a comparison, the skilled artisan can predict the importance of amino acid residues in a protein that correspond to amino acid residues important for activity or structure in similar proteins. One skilled in the art may opt for chemically similar amino acid substitutions for such predicted important amino acid residues.

One skilled in the art can also analyze the three-dimensional structure and amino acid sequence in relation to that structure in similar polypeptides. In view of such information, one skilled in the art may predict the alignment of amino acid residues of a polypeptide with respect to its three dimensional structure. In certain embodiments, one skilled in the art may choose to not make radical changes to amino acid residues predicted to be on the surface of the protein, since such residues may be involved in important interactions with other molecules. Moreover, one skilled in the art may generate test variants containing a single amino acid substitution at each desired amino acid residue. The variants can then be screened using activity assays known to those skilled in the art. Such variants could be used to gather information about suitable variants. For example, if one discovered that a change to a particular amino acid residue resulted in destroyed, undesirably reduced, or unsuitable activity, variants with such a change can be avoided. In other words, based on information gathered from such routine experiments, one skilled in the art can readily determine the amino acids where further substitutions should be avoided either alone or in combination with other mutations.

A number of scientific publications have been devoted to the prediction of secondary structure. See Moult, 1996, Curr. Op. in Biotech. 7:422-427; Chou et al, 1974, Biochemistry 13:222-245; Chou et al, 1974, Biochemistry 113:211-222; Chou et al, 1978, Adv. Enzymol Relat. Areas Mol. Biol 47:45-148; Chou et al, 1979, Ann. Rev. Biochem. 47:251-276; and Chou et al, 1979, Biophys. J. 26:367-384. Moreover, computer programs are currently available to assist with predicting secondary structure. One method of predicting secondary structure is based upon homology modeling. For example, two polypeptides or proteins that have a sequence identity of greater than about 30%, or similarity greater than 40% often have similar structural topologies. The recent growth of the protein structural database has provided enhanced predictability of secondary structure, including the potential number of folds within a polypeptide's or protein's structure. See Holm et al, 1999, Nucl. Acid. Res. 27:244-247. It has been suggested (Brenner et al, 1997, Curr. Op. Struct. Biol. 7:369-376) that there are a limited number of folds in a given polypeptide or protein and that once a critical number of 5 structures have been resolved, structural prediction will become dramatically more accurate.

Additional methods of predicting secondary structure include “threading” (Jones, 1997, Curr. Opin. Struct. Biol. 7:377-87; Sippl et al, 1996, Structure 4:15-19), “profile analysis” (Bowie et al, 1991, Science 253:164-170; Gribskov et al, 1990, Meth. Enzym. 183:146-159; Gribskov et al, 1987, Proc. Nat. Acad. Sci. 84:4355-4358), and “evolutionary linkage” (See Holm, 1999, supra; and Brenner, 1997, supra).

Certain embodiments of the invention are also described U.S. application Ser. No. 10/820,559, supra, and U.S. Pat. Nos. 6,130,318; 6,313,272; 6,335,426; 6,028,176; and related applications therein, including related priority documents and references, the entire contents of which are incorporated herein by reference.

In one embodiment, the modified IL-4 mutein receptor antagonists of the invention include a modification of replacing one or more amino acids occurring in the wtIL-4 protein at amino acid positions 121, 124 and/or 125 with another natural amino acid, for example, glutamic acid or aspartic acid, or any positively charged amino acid.

In another embodiment, the modified IL-4 mutein receptor antagonists of the invention include another modification of the N-terminus and C-terminus by deleting and/or inserting of one or more amino acids; and/or deletion of potential glycosylation sites therein. In one preferred embodiment, an N-terminus modification is an insertion of an amino acid, at amino acid position +2. In another preferred embodiment, a C-terminus modification is a deletion of at least one, at least two, at least three, at least four and at least five amino acids. However, deletions of greater than five amino acids from the C-terminus may affect the activity of the mhIL-4. Activity of the mhIL-4 from any of the modifications mentioned above and herein can be determined by using any of the methods described previously in related applications and/or patents, and methods described herein (e.g., the Bimolecular Interaction Analysis (BIA) and proliferative assays as described in U.S. application Ser. No. 10/820,559 (see Examples 4 and 5). The entire contents of U.S. application Ser. No. 10/820,559 is incorporated herein by reference.

In certain embodiments, protein variants include glycosylation variants wherein the number and/or type of glycosylation site has been altered compared to the amino acid sequences of the parent polypeptide. In certain embodiments, protein variants comprise a greater or a lesser number of N-linked glycosylation sites than the native protein. An N-linked glycosylation site is characterized by the sequence: Asn-X-Ser or Asn-X-Thr, wherein the amino acid residue designated as X may be any amino acid residue except proline. The substitution of amino acid residues to create this sequence provides a potential new site for the addition of an N-linked carbohydrate chain. Alternatively, substitutions that eliminate this sequence will remove an existing N-linked carbohydrate chain. Also provided is a rearrangement of N-linked carbohydrate chains wherein one or more N-linked glycosylation sites (typically those that are naturally occurring) are eliminated and one or more new N-linked sites are created.

Additional preferred variants include cysteine variants wherein one or more cysteine residues are added to, deleted from or substituted for another amino acid (e.g., serine) compared to the parent amino acid sequence. Cysteine variants may be useful when proteins must be refolded into a biologically active conformation such as after the isolation of insoluble inclusion bodies. Cysteine variants generally have fewer cysteine residues than the native protein, and typically have an even number to minimize interactions resulting from unpaired cysteines. In one embodiment of the invention, modified IL-4 mutein receptor antagonists of the invention includes cysteine residues are added to or near amino acid positions 38, 102 and/or 104.

In yet another embodiment of the invention, there is provided protein variants including mutations such as substitutions, additions, deletions, or any combination thereof, and are typically produced by site-directed mutagenesis using one or more mutagenic oligonucleotide(s) according to methods described herein, as well as according to methods known in the art (see, for example, Sambrook et al, MOLECULAR CLONING: A LABORATORY MANUAL, 3rd Ed., 2001, Cold Spring Harbor, N.Y. and Berger and Kimmel, METHODS IN ENZYMOLOGY, Volume 152, Guide to Molecular Cloning Techniques, 1987, Academic Press, Inc., San Diego, Calif., which are incorporated herein by reference).

According to certain embodiments, amino acid substitutions are those that: (1) reduce susceptibility to proteolysis, (2) reduce susceptibility to oxidation, (3) alter binding affinity for forming protein complexes, (4) alter binding affinities, and/or (5) confer or modify other physicochemical or functional properties on such polypeptides. According to certain embodiments, single or multiple amino acid substitutions (in certain embodiments, conservative amino acid substitutions) may be made in the naturally occurring sequence (in certain embodiments, in the portion of the polypeptide outside the domain(s) forming intermolecular contacts).

In preferred embodiments, a conservative amino acid substitution typically does not substantially change the structural characteristics of the nucleotide sequence (e.g., a replacement amino acid should not tend to break a helix that occurs in the nucleotide sequence, or disrupt other types of secondary structure that characterizes the nucleotide sequence). Examples of art-recognized polypeptide secondary and tertiary structures are described in PROTEINS, STRUCTURES AND MOLECULAR PRINCIPLES, (Creighton, Ed.), 1984, W. H. Freeman and Company, New York; INTRODUCTION TO PROTEIN STRUCTURE (C. Branden and J. Tooze, eds.), 1991, Garland Publishing, New York, N.Y.; and Thornton et al., 1991, Nature 354:105, each of which are incorporated herein by reference.

Peptide analogs are commonly used in the pharmaceutical industry as non-peptide drugs with properties analogous to those of the template peptide. These types of non-peptide compound are termed “peptide mimetics” or “peptidomimetics”. See Fauchere, 1986, Adv. Drug Res. 15:29; Veber & Freidinger, 1985, TINS p. 392; and Evans et al., 1987, J. Med. Chem. 30:1229, which are incorporated herein by reference for any purpose. Such compounds are often developed with the aid of computerized molecular modeling. Peptide mimetics that are structurally similar to therapeutically useful peptides may be used to produce a similar therapeutic or prophylactic effect. Generally, peptidomimetics are structurally similar to a paradigm polypeptide (i.e., a polypeptide that has a biochemical property or pharmacological activity), such as human antibody, but have one or more peptide linkages optionally replaced by a linkage selected from: —CH2—NH—, —CH2—S—, —CH2—CH2—, —CH═CH— (cis and trans), —COCH2—, —CH(OH)CH2—, and —CH2SO—, by methods well known in the art. Systematic substitution of one or more amino acids of a consensus sequence with a D-amino acid of the same type (e.g., D-lysine in place of L-lysine) may be used in certain embodiments to generate more stable peptides. In addition, constrained peptides comprising a consensus sequence or a substantially identical consensus sequence variation may be generated by methods known in the art (Rizo & Gierasch, 1992, Ann. Rev. Biochem. 61:387, incorporated herein by reference for any purpose); for example, by adding internal cysteine residues capable of forming intramolecular disulfide bridges which cyclize the peptide.

Still in another preferred embodiment, modified IL-4 mutein receptor antagonists as used herein includes the IL-4RA mutein described in U.S. Pat. Nos. 6,028,176 and 6,313,272, the entire contents of which are incorporated herein by reference. The modified IL-4 mutein receptor antagonists as described herein include those polypeptides and functional fragments thereof with additional amino acid substitutions, including those substitutions which enable the site-specific coupling of at least one non-protein polymer, such as polypropylene glycol, polyoxyalkylene, or polyethylene glycol (PEG) molecule to the mutein. Site-specific coupling of PEG, for example, allows the generation of a modified mutein which possesses the benefits of a polyethylene-glycosylated (PEGylated) molecule, namely increased plasma half life (e.g., at least 2 to 10-fold greater, or 10 to 100-fold greater than that of unmodified IL4RA) while maintaining greater potency over non-specific PEGylation strategies such as N-terminal and lysine side-chain PEGylation. Methods providing for efficient PEGylation are described in U.S. application Ser. No. 10/820,559, which is incorporated herein by reference.

The IL-4 mutein must be purified properly to allow efficient PEGylation. Purification is described U.S. application Ser. No. 10/820,559 (see Example 2). For example, if the mutein is refolded in the presence of a sulfhydryl protecting agent such as beta-mercaptoethanol, glutathione, or cysteine, the purified mutein cannot be PEGylated because the active sulhydryl in the introduced cysteine on IL-4 is inactivated by the oxidized protecting agent. A covalent disulfide bond is formed between the IL-4 mutein's free cysteine and the protecting agent. In contrast, the use of the sulfhydryl protecting agent dithiothreitol (DTT), which oxidizes to form a stable disulfide bond, will not form a covalent bond with the IL-4 mutein's free cysteine, thus leaving its sulfydryl group free to react with the PEG maleimide reagent. IL-4 muteins purified after refolding in the presence of beta-mercaptoethanol, glutathione, or cysteine can react with the PEG reagent if treated with DTT, but a mixture of monoPEGylated and multiPEGylated products are generated, suggesting that existing IL-4 cysteines are also PEGylated. PEGylation of existing cysteines would lead to misfolded products that are inactive.

The Ki of modified IL-4 mutein receptor antagonists to the IL-4 receptor can be assayed using any method known in the art, including technologies such as real-time Bimolecular Interaction Analysis (BIA) as described in U.S. application Ser. No. 10/820,559 (see Example 4). The capacity of modified IL-4 mutein receptor antagonists to inhibit the proliferative response of immune cells can be assessed using proliferative assays as described in U.S. application Ser. No. 10/820,559 (see Example 5).

A number of modified IL-4 mutein receptor antagonists with the characteristics described above have been identified in U.S. application Ser. No. 10/820,559, incorporated herein by reference, by screening candidates with the above assays. In one embodiment, a non-protein polymer (e.g., polyethylene glycol) is coupled to at least amino acid residue positions 38, 102 and/or 104.

Also inherent in this invention is the selection of the specific site of amino acid substitution which enables proper folding of the polypeptide following expression. Modified IL-4 mutein receptor antagonists bind to IL-4 and IL-13 with an affinity loss not greater than 10-fold relative to that of EL-4RA. Modified IL-4 mutein receptor antagonists inhibit IL-4 and IL-13 mediated activity with a loss of potency not greater than 10-fold relative to that of IL-4RA. In addition, modified IL-4 mutein receptor antagonists possess a plasma half-life which is at least 2 to 10-fold greater than that of unmodified IL-4RA.

The above polypeptide variants are illustrative of the types of modified human IL-4 polypeptides to be used in the methods claimed herein, but are not exhaustive of the types of variations of the claimed invention which may be embodied by the invention. Derivatives of the above polypeptide which fit the criteria of the claims should also be considered. All of the polypeptides and functional fragments thereof can be screened for efficacy following the methods taught herein and in the examples.

In one preferred embodiment of the invention, there is provided a modified human IL-4 mutein antagonist termed “mhIL-4.” MhIL-4 and its derivatives thereof are accomplished in wide variety of host cells common for use in recombinant technology. Host cells suitable for the recombinant production of mhIL-4 are known to those skilled in the art including prokaryotic cells such as strains of E. coli, Bacillus or Pseudomonas (Kung, H.-F., M. Boublik, V. Manne, S. Yamazaki and E. Garcia, Curr. Topics in Cell. Reg. 26: 531-542, 1995) or unicellular eukaryotic cells as yeast Saccharomyces cerevisiae (Bemis, L. T., F. J. Geske and R. Strange, Methods Cell Biol., 46: 139-151, 1995). Host cells for recombinant production may also be derived from multicellular eukaryotes comprising invertebrates as insects (Spodoptera frugiperda Sf9 cells) (Altmann F., E. Staudacher, I B Wilson, and L Marz, Glycoconj J., 16: 109-123, 1999) and vertebrate cells, including numerous mammalian cell lines comprising mouse fibroblasts, Chinese hamster ovary cells (CHO/-DHFR) (Urlaub and Chasin, Proc Nat Acad Sci 77:4216, 1980), baby hamster kidney (9BHK, ATCC CCL 10); monkey kidney CV1 line transformed by SV40 (COS-7, ATCC CRL 1651) and human embryonic kidney cell line 293 (Tartaglia et al., Proc Nat Acad Sci 88: 9292-9296, 1991 and Pennica et al., J. Biol. Chem. 267: 21172-21178, 1992).

Although bacteria and yeast have been the standard recombinant host cells for many recombinant polypeptides, including mhIL-4, recently transformed cells from higher plants to express human recombinant proteins as antibodies (Hiatt, A. T. and J. K. Ma, Int. Rev. Immunol., 10: 139-152, 1993) and hemoglobin (Theisen, M. in Chemicals Via Higher Plant Bioengineering, F. Shahidi et al., eds, Plenum Publishers, NY, p. 211-220, 1999). Plant biotechnology offers many advantages for efficient production of heterologous polypeptides, and this approach may be useful for production of the modified human IL-4 receptor antagonists described herein, including mhIL-4 and its derivatives (see also Plant Technology: New Products and Applications, John Hammond, et al., eds., Springer, N.Y., 1999). The appropriate choice of host cell is determined by what is efficient and required for the accurate expression, processing and recovery of monomer SP-B1-15.

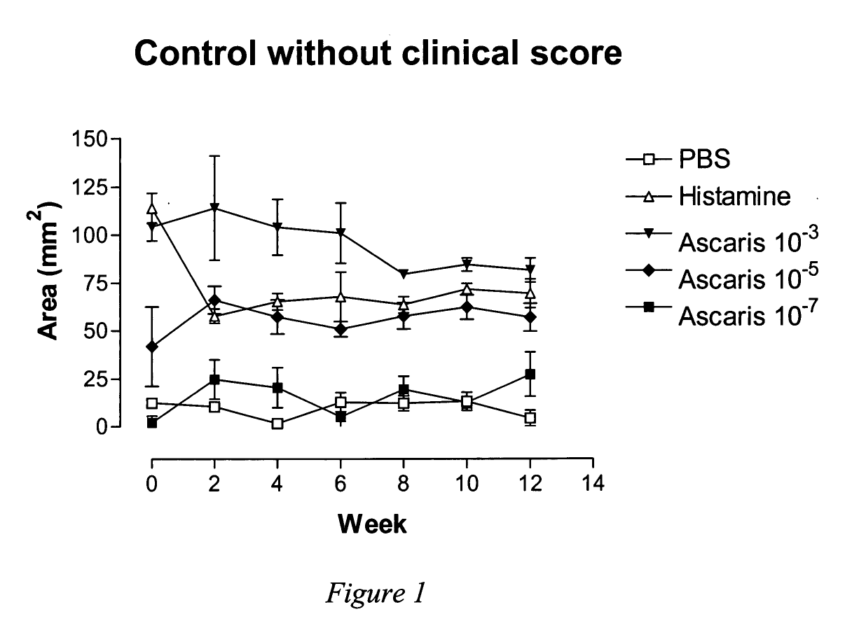

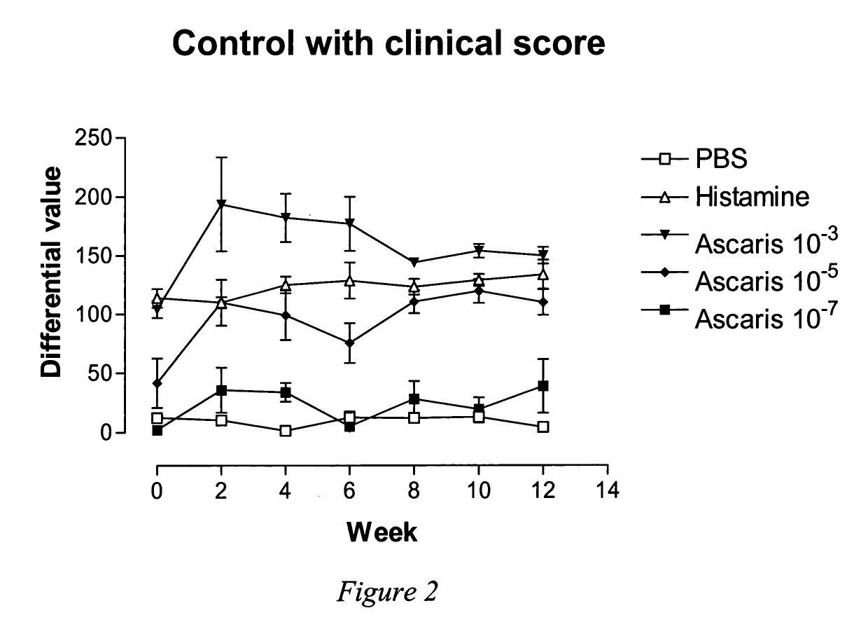

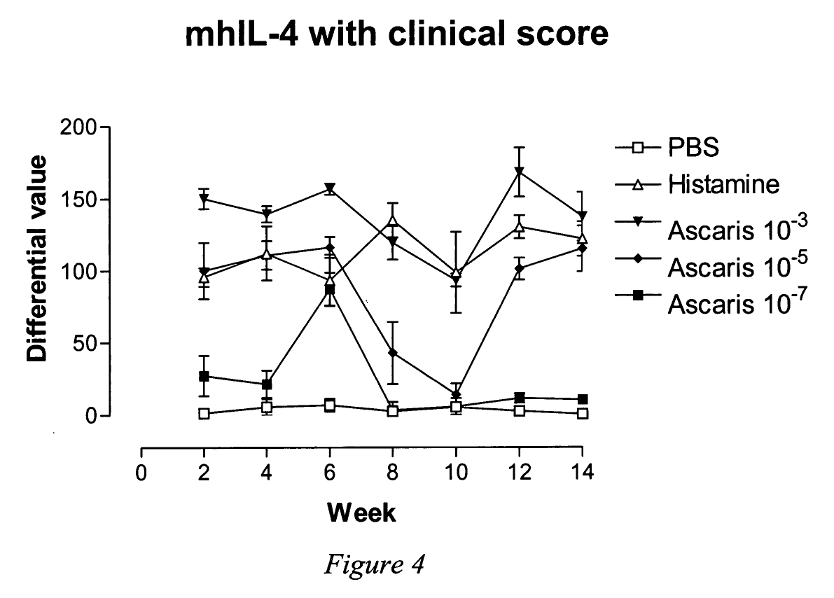

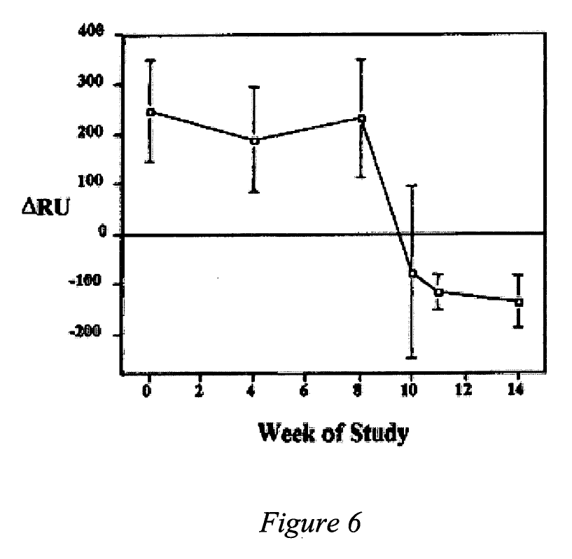

In one preferred embodiment of the invention, there is provided a method of administering a therapeutically effective amount of modified human IL-4 receptor antagonist, including mhIL-4, to a subject to ameliorate symptoms associated with dermatitis (see Example 1). Studies administering mhIL-4 to cynomolgus monkeys showed that animals challenged with various antigens including histamine, PBS and Ascaris suum and receiving about 1 mg/kg of mhIL-4, 3× a week, subcutaneously (sc) for 16 weeks (see Example 1) had a reduce wheal-flare response by the eighth week as measured by skins tests and by reduced levels of IgE (see FIGS. 1-5). There were also no significant changes in the wheal and flare response to intradermal injections of histamine or PBS throughout the study (see FIGS. 1 and 2). However, in those animals receiving mhIL-4, after the twelfth week, there was a return of the wheal-flare response. The return of the wheal flare response suggests loss of activity or protection in vivo, and is likely due to an inhibitory substance or agent binding to mhIL—and preventing mhIL—from binding to its receptor (see FIG. 6). Data suggest that the inhibitory substance is an antibody because loss of activity is observed by an increase in IgE levels at about the same time, or about the twelfth week (sees FIG. 4). Hence, the return of the wheal-flare response may be a species-dependent effect, since mhIL-is derived from a human recombinant protein (i.e. human IL-4). That is, systemic or local, e.g., subcutaneous injection, of human derived protein into a non-human subject may give rise to the higher immune response observed in the monkey study.

In another embodiment, there is provided a method of administering a therapeutically effective amount of modified human IL-4 receptor antagonist, including mhIL-4, to a human subject to ameliorate symptoms associated with dermatitis, for example, atopic dermatitis. In one preferred embodiment, about 25 mg, or about 0.4 mg/kg, is administered systemically or locally, preferably, subcutaneously once a day for up to about twenty eight days, or about four weeks. The immune response will be monitored similar to the monkey study by making daily observation of the existing dermatitis and plasma levels of IgE before and after the completion of dosing. Similar to the monkey study appropriate controls will be in performed, hence some patients will not receive the drug. It is anticipated that in those patients receiving the drug, there will be a pronounced reduction in the immune response as compared to the monkey study. Upon reduction of the immune response, patients will be taken off the drug, or mhIL-4, and monitored for the length of remission. Thus, administration of mhIL-4 to these patients is expected to reduce or eliminate early and/or latent immune responses.

Also, administration of mhIL-4 to human subjects should produce a reduced immune response because fewer antibodies against the antagonists are expected. Fewer antibodies to mhIL-4 and its epitopes imply that there are fewer inhibiting substances or agents binding to mhIL-4. Fewer inhibiting substances and agents binding directly or indirectly to mhIL-4 allows the drug to bind to the IL-4 receptor and thus inhibit the IL-4 and IL-13 induced response and inactivating the cascade of downstream events, e.g. release of various interleukins, chemokines, and chemoattractants involved in an immune response.

Although the invention describes various dosages, it will be understood by one skilled in the art that the specific dose level and frequency of dosage for any particular subject in need of treatment may be varied and will depend upon a variety of factors. These factors include the activity of the specific polypeptide or functional fragment thereof, or functional fragment thereof, the metabolic stability and length of action of that compound, the age, body weight, general health, sex, diet, mode and time of administration, rate of excretion, drug combination, the severity of the particular condition, and the host undergoing therapy. Generally, however, dosage will approximate that which is typical for known methods of administration of the specific compound. For example, for administration of mhIL-4, an approximate dosage would be about 0.2 mg/kg, about 0.3 mg/kg, about 0.4 mg/kg, about 0.5 mg/kg, about 0.6 mg/kg or greater, to a human subject, and about 1 mg/kg to a non-human, or animal subject. In another preferred embodiment, for a subcutaneous administration of mhIL-4 to a human subject, an appropriate amount may be more about 0.4 mg/kg. Hence, an appropriate amount can be determined by one of ordinary skill in the art and using routine procedures such as those provided herein (see Example 1).

The compositions and formulations of the invention can be administered systemically or locally. The local composition format can be selected from the group consisting of an aerosol (e.g., nebulization, dry powder or metered dose inhalation), a drop, a spray, a cream, and an ointment. Also, Depending on the format, the compositions can include other carrier agents including swelling mucoadhesive particulates, pH sensitive microparticulates, nanoparticles/latex systems, ion-exchange resins and other polymeric gels and agents (Ocusert, Alza Corp., California; Joshi, A., S. Ping and K. J. Himmelstein, Patent Application WO 91/19481). These agents and systems maintain prolonged drug contact with the absorptive surface preventing washout and nonproductive drug loss.

The compositions of the invention can also have formulations whereby the modified human IL-4 receptor antagonists are in a delayed-released format. Suitable examples of preparations having a delayed release are, for example, semi-permeable matrices consisting of solid hydrophobic polymers which contain the protein; these matrices are shaped articles, for example film tablets or microcapsules. Examples of matrices having a delayed release are polyesters, hydrogels [e.g. poly(2-hydroxyethyl methacrylate)—described by Langer et al., J. Biomed. Mater. Res., 15:167-277 [1981] and Langer, Chem. Tech., 12:98-105 [1982]—or poly(vinyl alcohol)], polyactides (U.S. Pat. No. 3,773,919, EP 58,481), copolymers of L-glutamic acid and gamma-ethyl-L-glutamate (Sidman et al., Biopolymers, 22:547-556 [1983]), non-degradable ethylene/vinyl acetate (Langer et al., loc. sit.), degradable lactic acid/glycolic acid copolymers such as Lupron Depot™ (injectable microspheres consisting of lactic acid/glycolic acid copolymer and leuprolide acetate) and poly-D-(−)-3-hydroxybutyric acid (EP 133,988). While polymers such as ethylene/vinyl acetate and lactic acid/glycolic acid enable the molecules to be released for periods of greater than 100 days, the proteins are released over relatively short periods of time in the case of some hydrogels. If encapsulated proteins remain in the body over relatively long periods of time, they can then be denatured or aggregated by moisture at 37° C., resulting in a loss of biological activity and possible changes in immunogenicity. Meaningful strategies for stabilizing the proteins can be developed, depending on the mechanism involved. If it is found, for example, that the mechanism which leads to the aggregation is based on intermolecular S-S bridge formation as a result of thiodisulphide exchange, stabilization can be achieved by modifying the sulphydryl radicals, lyophilizing from acid solutions, controlling the moisture content, using suitable additives and developing special polymer/matrix compositions.

The formulations of the invention exhibiting delayed release also include modified human IL-4 receptor antagonists which are enclosed in liposomes. IL-4 antagonist-containing liposomes are prepared by methods which are known per se: DE 3,218,121; Epstein et al., Proc. Natl. Acad. Sci. USA, 82;3688-3692 (1985); Hwang et al., Proc. Natl. Acad. Sci. USA, 77:4030-4034 (1980); EP 52,322; EP 36,676; EP 88,046; EP 143,949; EP 142,641; Japanese Patent Application 83-118008; U.S. Pat. Nos. 4,485,045 and 4,544,545; and also EP 102,324. As a rule, the liposomes are of the small (approximately 200-800 Angstrom) unilamellar type having a lipid content of greater than approximately 30 mol % cholesterol, with the proportion in each case being adjusted for the optimum IL-4 antagonists. Liposomes exhibiting an extended circulation time are disclosed in U.S. Pat. No. 5,013,556.

Also contemplated is use of the DNA sequences encoding the IL-4 muteins of this invention in gene therapy applications. Gene therapy applications contemplated include treatment of those diseases in which IL-4 is expected to provide an effective therapy due to its immunomodulatory activity, e.g., Multiple Sclerosis (MS), Insulin-dependent Diabetes Mellitus (IDDM), Rheumatoid Arthritis (RA), Systemic Lupus Erythematosus (SLE), uveitis, orchitis, primary biliary cirrhosis, malaria, leprosy, Lyme Disease, atopic dermatitis, contact dermatitis, psoriasis, B cell lymphoma, acute lymphoblastic leukemia, non-Hodgkins lymphoma, cancer, osteoarthritis and diseases that are otherwise responsive to IL-4 or infectious agents sensitive to IL-4-mediated immune response.

Local delivery of IL-4 muteins using gene therapy may provide the therapeutic agent to the target area. Both in vitro and in vivo gene therapy methodologies are contemplated. Several methods for transferring potentially therapeutic genes to defined cell populations are known. See, e.g., Mulligan, “The Basic Science Of Gene Therapy”, Science, 260: 926-31 (1993). These methods include:
1) Direct gene transfer. See, e.g., Wolff et al, “Direct Gene transfer Into Mouse Muscle In Vivo”, Science. 247:1465-68 (1990);
2) Liposome-mediated DNA transfer. See, e.g., Caplen at al., “Liposome-mediated CFTR Gene Transfer To The Nasal Epithelium Of Patients With Cystic Fibrosis”, Nature Med. 3: 39-46 (1995); Crystal, “The Gene As A Drug”, Nature Med. 1:15-17 (1995); Gao and Huang, “A Novel Cationic Liposome Reagent For Efficient Transfection Of Mammalian Cells”, Biochem. Biophys. Res. Comm., 179:280-85 (1991);
3) Retrovirus-mediated DNA transfer. See, e.g., Kay et al., “In Vivo Gene Therapy Of Hemophilia B: Sustained Partial Correction In Factor IX-Deficient Dogs”, Science, 262:117-19 (1993); Anderson, “Human Gene Therapy”, Science 256:808-13 (1992).
4) DNA Virus-mediated DNA transfer. Such DNA viruses include adenoviruses (preferably Ad-2 or Ad-5 based vectors), herpes viruses (preferably herpes simplex virus based vectors), and parvoviruses (preferably “defective” or non-autonomous parvovirus based vectors, more preferably adeno-associated virus based vectors, most preferably AAV-2 based vectors). See, e.g., Ali et al., “The Use Of DNA Viruses As Vectors For Gene Therapy”, Gene Therapy, 1:367-84 (1994); U.S. Pat. No. 4,797,368, incorporated herein by reference, and U.S. Pat. No. 5,139,941, incorporated herein by reference.

The choice of a particular vector system for transferring the gene of interest will depend on a variety of factors. One important factor is the nature of the target cell population. Although retroviral vectors have been extensively studied and used in a number of gene therapy applications, these vectors are generally unsuited for infecting non-dividing cells. In addition, retroviruses have the potential for oncogenicity.

Adenoviruses have the advantage that they have a broad host range, can infect quiescent or terminally differentiated cells, such as neurons or hepatocytes, and appear essentially non-oncogenic. See, e.g., Ali et al., supra. Adenoviruses do not appear to integrate into the host genome. Because they exist extrachromosomally, the risk of insertional mutagenesis is greatly reduced. Ali et al., supra, p. 373.

Adeno-associated viruses exhibit similar advantages as adenoviral-based vectors. However, AAVs exhibit site-specific integration on human chromosome 19. Ali et al., supra, p. 377.

According to this embodiment, gene therapy with DNA encoding the IL-4 muteins of this invention is provided to a patient in need thereof, concurrent with, or immediately after diagnosis.

This approach takes advantage of the selective activity of the IL-4 muteins of this invention to prevent undesired autoimmune stimulation. The skilled artisan will appreciate that any suitable gene therapy vector containing IL-4 mutein DNA may be used in accordance with this embodiment. The techniques for constructing such a vector are known in the art.

The present invention is more particularly described in the following examples which are intended as illustrative only since numerous modifications and variations therein will be apparent to those skilled in the art. The following examples are intended to illustrate but not limit the invention.

### EXAMPLE 1

### MhIL-4 Antagonist Causes Transient Inhibition of Antigen-Induced Cutaneous Response in Monkeys

Materials and Methods

The animals used in this study were adult male cynomolgus monkeys (Macaca fascicularis) weighing between about 5.0 to about 8.0 kg. All animals demonstrated naturally occurring skin sensitivity to Ascaris suum, a parasitic nematode. Each animal was housed individually in open mesh squeeze cages with food provided daily and water available ad libitum. Room temperature and humidity were held constant at 22° C. and 75%, respectively, and a 12 hour light-dark cycle was imposed beginning at 6:00 am. All animals were fasted approximately 12 hours prior to the study. For each study all animals were anesthetized with ketamine hydrochloride (7.0 mg/kg, i.m.; Ketaset, Fort Dodge, Iowa) and xylazine (1.2 mg/kg, i.m.; Bayer Corp., Elkart, Ind.) and supplemented with ketamine alone (5 mg/kg, i.m.) as needed.

Eight cynomolgus monkeys with no history of previous exposure to mhIL-4 were used to study the effect of mhIL-4 on Ascaris suum induced wheal and flare reactions. The monkeys abdomen and chests were initially injected (40 μL, i.d.) with histamine (0.25 mg/mL), phosphate buffered saline (PBS) and Ascaris suum (10⁻³, 10⁻⁵, 10⁻⁷ g/mL). Subsequently, four monkeys received mhIL-4 at 1 mg/kg, s.c, 3 times per week for 12 weeks, while the other four animals received PBS at the same dosing regime. Wheal and flare responses as well as clinical scores (1.0 no reaction, 1.5 pink and raised and 2.0 red and raised) were determined prior to administration of mhIL-4 and immediately after all challenge injections, and again 15-20 minutes post injection. All eight animals were skin tested (measurement of wheal size) biweekly. The study spanned a five month period. The Institute for Animal Care and Use Committee at Bayer Corporation approved the protocol (#96-230).

A baseline response was established by performing skin tests on weeks 0 and 2 on all animals in the treatment group prior to dosing with mhIL-4. Dosing began on week 2 and continued 3 times per week for 12 weeks. All animals had blood drawn (4.5 mL) for IgE and T cell activation analysis prior to each skin test. All skin tests and blood draws were at least 72 hours after the previous dose.

Injection of Antigen

Prior to injection, the chest and abdominal area was cleaned with 70% ethyl alcohol and shaved. Intradermal injections of 40 μL of antigen (10⁻³, 10⁻⁵, 10⁻⁷ μg/mL) Ascaris suum extract (Greer Labs. Inc., Lenoir, N.C.), PBS (Gibco BRL, Grand Island, N.Y.), and histamine dihydrochloride (0.25 mg/mL, Sigma Chemical Co., St. Louis, Mo.) were subsequently administered to each animal. All solutions were prepared freshly on the day the tests were performed. Ascaris suum was diluted in PBS 1:100 serially from 10⁻¹ μg/mL (Greer Labs) stock solution, while the histamine was made by adding 10 mg (Sigma Labs, H-7250) to 40 mL PBS then further diluting 1:10 in PBS.

Measurement of Wheal and Flare and Clinical Scoring

Wheal and flare responses were measured using vernier calipers on perpendicular axis at two time points, 0 minutes and 15 to 20 minutes after the last injection for each animal. A clinical score of 1.0 (PBS), 1.5 (pink and raised) or 2.0 (red and raised) were assigned immediately and 15 to 20 minutes after each animals injections. FIGS. 1-4 are representative graphs showing these types of measurements (with and without clinical score).

Induced Cutaneous Wheal and Flare Response

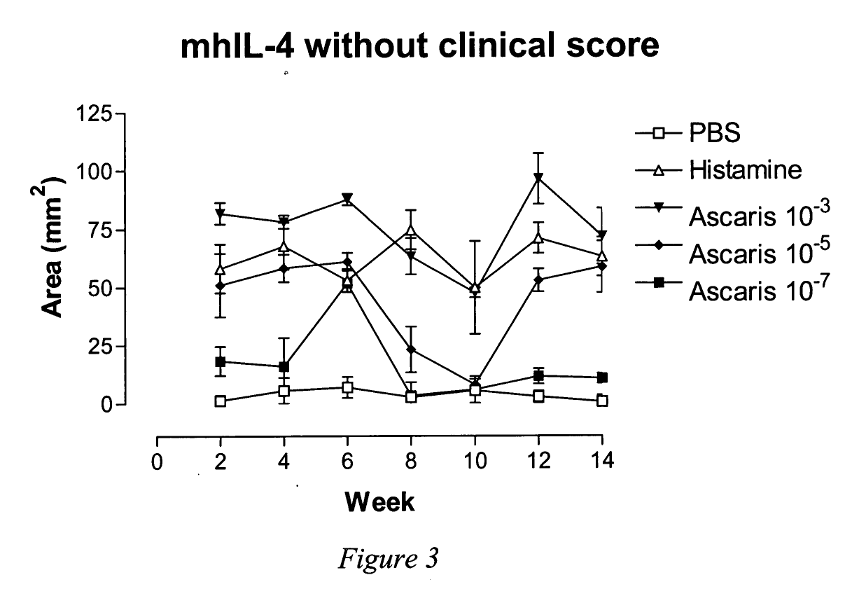

The summary of data without clinical score (area in mm²) was determined by subtracting the first measurement of area taken immediately after the subcutaneous injections from that of the second measurement of area taken 15 to 20 minutes later. Values from each group were averaged, and standard deviations as well as SEM were calculated. The same method was used to calculate the data with the clinical score (FIGS. 1 and 3), except these areas were multiplied by the clinical score prior to determining the final values (FIGS. 2 and 4).

Plasma Surface Resonance (Biacore Assay)

Binding of mhIL-4 to IL-4 receptor-α was monitored by surface plasmon resonance using a BIAcore 2000 instrument (Biacore, Inc., Piscataway N.J.). An NTA chip was charged with Ni2+ by injecting a solution of 100 mM NiCl2 (Sigma, St. Louis, Mo.) at a flow rate of 10 μL/min for 3 minutes. A total of 180-200 resonance units (RU) were bound. His-tagged soluble IL-4 receptor was diluted in HBS buffer (Biacore, Inc.) containing 5 mM EDTA and 0.005% surfactant P-20 to 100 ng/ml and injected for 6 minutes using a flow rate of 5 μL/min. The resulting RU increase was in the range of 800 to 1000, which corresponds to a surface concentration of 1 ng/mm2 or 270 mM of IL4R. Cynomolgus monkey plasma samples were diluted 1:5 in HBS buffer, spiked with either 0 (controls) or 100 ng on mhIL-4 and injected onto the chip surface containing captured IL-4R at 10 μL/min for 5 minutes. Surface regeneration was accomplished after each binding experiment with 20 μL of 500 mM EDTA (Sigma) at 10 μL/min followed by 20 μL of 0.1% SDS (Sigma). FIG. 6 is representative of this type of assay.

Results

Treatment with mhIL-4 caused a total, but transient, inhibition of antigen-induced cutaneous response for the lower Ascaris suum concentrations (10⁻⁵ & 10⁻⁷) as shown from the size of the wheal response (see FIGS. 3 and 4 and Tables I and II). FIGS. 3 and 4 and Tables I and II show that the reduction in cutaneous response was evident at week 8 of the study (i.e., after 6 weeks of treatment) and peaked at week 10 (p<0.05 vs. week 6 at all doses of Ascaris suum). However, despite continued treatment with mhIL-4, the wheal response to cutaneous antigen injection returned to baseline (pretreatment) levels. Histamine was used as a non antigen stimulated positive control.

This is compared with the control animals who received PBS injections instead of mhIL-4 (see FIGS. 1 and 2 and Tables I and II). FIGS. 1 and 2 and Tables I and II show that there is a constant reaction to all doses of Ascaris suum and histamine. The PBS data showed a baseline response as a negative control. FIG. 2 shows a similar response to all administered compounds (PBS, Ascaris suum and histamine), however, the values are increased by the clinical score factor (see Materials and Methods). In summary, vehicle control studies demonstrate that continued treatment with PBS had no effect on antigen-induced cutaneous wheal responses throughout the study (see FIGS. 1 & 2).

TABLE I
Summary of wheal response measurements

| | | | | | | | | | | | | | | Week | |
|---|---|---|---|---|---|---|---|---|---|---|---|---|---|---|---|
| Week 0 | SEM | Week 2 | SEM | Week 4 | SEM | Week 6 | SEM | Week 8 | SEM | Week 10 | SEM | Week 12 | SEM | 14 | SEM |

Skin Test Bay 16-9996 Summarized Values without Clinical Score (Area)

| | | | | | | | | | | | | | | | | |
|---|---|---|---|---|---|---|---|---|---|---|---|---|---|---|---|---|
| PBS | 9.6 | 2.1 | 1.3 | 2.2 | 5.6 | 5.6 | 7 | 4.5 | 2.7 | 6.4 | 5.6 | 1.6 | 2.9 | 2.5 | 0.8 | 2.8 |
| Histamine | 136.9 | 12.8 | 58.2 | 10.5 | 67.9 | 10.1 | 53.1 | 5 | 74.7 | 8.3 | 49.9 | 20 | 71.1 | 6.5 | 63.2 | 8.3 |
| Ascaris 10-3 | 94.6 | 10.8 | 81.8 | 4.6 | 78.2 | 2.8 | 87.7 | 2.4 | 63.4 | 7.8 | 47.8 | 2.1 | 96.5 | 10.9 | 71.9 | 11.9 |
| Ascaris 10-5 | 55.5 | 10.6 | 51.1 | 13.7 | 58.4 | 5.9 | 61.2 | 3.8 | 23.3 | 9.9 | 8.2 | 2.4 | 53.1 | 5 | 58.8 | 11 |
| Ascaris 10-7 | 19.2 | 9.6 | 18.4 | 6.3 | 16 | 12.4 | 51.9 | 1.4 | 3.4 | 1.2 | 6 | 5.8 | 11.7 | 3.3 | 10.8 | 2.1 |

Skin Test Bay 16-9996 Summarized Values with Clinical Score

| | | | | | | | | | | | | | | | | |
|---|---|---|---|---|---|---|---|---|---|---|---|---|---|---|---|---|
| PBS | 9.6 | 2.1 | 1.3 | 2.2 | 5.6 | 5.6 | 7 | 4.5 | 2.7 | 6.4 | 5.6 | 1.6 | 2.9 | 2.5 | 0.8 | 2.8 |
| Histamine | 229.5 | 18.2 | 95.8 | 6.4 | 112.4 | 18.8 | 93.7 | 17.8 | 135.4 | 11.9 | 99.1 | 28 | 130.7 | 8 | 122.4 | 11.9 |
| Ascaris 10-3 | 165.1 | 16.1 | 150.3 | 7 | 139.6 | 5.7 | 156.9 | 3.9 | 119.7 | 11.7 | 93.7 | 4.6 | 168.5 | 16.9 | 137.9 | 16.8 |
| Ascaris 10-5 | 91.8 | 10.1 | 100.3 | 19.4 | 111.1 | 9.9 | 116.6 | 7.2 | 43.2 | 21.6 | 14.4 | 7.5 | 101.7 | 7.7 | 115.6 | 15.8 |
| Ascaris 10-7 | 19.2 | 9.6 | 27.3 | 13.9 | 21.6 | 9.5 | 87.6 | 11.3 | 3.4 | 1.2 | 6 | 5.8 | 11.7 | 3.3 | 10.8 | 2.1 |

| | | | | | | | | | | | | | |
|---|---|---|---|---|---|---|---|---|---|---|---|---|---|
| Week 0 | SEM | Week 2 | SEM | Week 4 | SEM | Week 6 | SEM | Week 8 | SEM | Week 10 | SEM | Week 12 | SEM |

Skin Test Vehicle Control (PBS) Summarized Values without Clinical Score (Area)

| | | | | | | | | | | | | | | |
|---|---|---|---|---|---|---|---|---|---|---|---|---|---|---|
| PBS | 12.3 | 2.5 | 10.4 | 1.7 | 1.6 | 2.5 | 12.7 | 5 | 12.1 | 4 | 13.1 | 4.7 | 4.4 | 4.2 |
| Histamine | 113.9 | 7.9 | 57.6 | 3.8 | 65.2 | 4.3 | 67.7 | 12.9 | 63.5 | 4.2 | 71.6 | 3.1 | 69.4 | 7.5 |
| Ascaris 10-3 | 104.2 | 7.3 | 114 | 27.1 | 104 | 14.5 | 100.9 | 15.7 | 79.5 | 1.6 | 84.5 | 3.4 | 81.6 | 6.3 |
| Ascaris 10-5 | 41.8 | 20.8 | 66 | 7.2 | 57.2 | 9 | 50.8 | 4 | 57.6 | 6.9 | 62.3 | 6.4 | 57 | 7.3 |
| Ascaris 10-7 | 2.1 | 3.4 | 24.6 | 10.2 | 20.3 | 10.5 | 5.1 | 7 | 19.4 | 6.8 | 12.5 | 3 | 27.2 | 11.5 |

Skin Test Vehicle Control (PBS) Summarized Values with Clinical Score

| | | | | | | | | | | | | | | |
|---|---|---|---|---|---|---|---|---|---|---|---|---|---|---|
| PBS | 12.3 | 2.5 | 10.4 | 1.7 | 1.5 | 2.5 | 12.7 | 5 | 12.1 | 4 | 13.1 | 4.7 | 4.4 | 4.2 |
| Histamine | 113.9 | 7.9 | 110 | 4.6 | 125.2 | 7.1 | 128.8 | 15.3 | 123.4 | 6.7 | 129.2 | 5.1 | 134 | 12.3 |
| Ascaris 10-3 | 104.2 | 7.3 | 193.7 | 39.8 | 182.3 | 20.5 | 177.1 | 23 | 144.2 | 2.2 | 154 | 5.8 | 150.2 | 7.1 |
| Ascaris 10-5 | 41.8 | 20.8 | 110.1 | 19.7 | 99.3 | 21.2 | 75.5 | 17 | 110.8 | 9.9 | 120 | 10.2 | 110.3 | 10.8 |
| Ascaris 10-7 | 2.1 | 3.4 | 35.8 | 19 | 33.9 | 7.9 | 5.1 | 7 | 28.1 | 15.1 | 19.6 | 9.8 | 39 | 22.7 |

TABLE II
Individual animal data (weeks 0-6)

| | Week 0 | | | Week 2 | | | Week 4 | | | Week 6 | | |
|---|---|---|---|---|---|---|---|---|---|---|---|---|
| | Wheal | Clinical | | Wheal | Clinical | | Wheal | Clinical | | Wheal | Clinical | |
| Animal # | (mm²) | Score | Total | (mm²) | Score | Total | (mm²) | Score | Total | (mm²) | Score | Total |

Control Study

| | | | | | | | | | | | | | |
|---|---|---|---|---|---|---|---|---|---|---|---|---|---|
| 93-282 | PBS | 61.8 | 1 | 58.1 | 70.2 | 1 | 70.2 | 60.3 | 1 | 60.3 | 61.5 | 1 | 61.5 |
| | His | 116.6 | 1.5 | 175 | 102.8 | 1.5 | 154.3 | 111.4 | 1.5 | 167 | 128.4 | 1.5 | 192.6 |
| | Asc 10⁻³ | 94.1 | 1.5 | 141.1 | 108.6 | 1.5 | 162.8 | 146.3 | 1.5 | 219.5 | 136.4 | 1.5 | 204.6 |
| | Asc 10⁻⁵ | 52.8 | 1 | 52.8 | 90.7 | 1.5 | 136.1 | 86.3 | 1.5 | 129.4 | 92.4 | 1 | 92.4 |
| | Asc 10⁻⁷ | 51.8 | 1 | 51.8 | 38.6 | 1 | 38.6 | 61 | 1.5 | 91.5 | 54.9 | 1 | 54.9 |
| 43-53 | PBS | 59.3 | 1 | 59.3 | 57.8 | 1 | 57.8 | 68.9 | 1 | 68.9 | 55.5 | 1 | 55.6 |
| | His | 106.2 | 1.5 | 162.4 | 111.6 | 1.5 | 167.3 | 139.9 | 1.5 | 209.9 | 101.2 | 1.5 | 151.8 |
| | Asc 10⁻³ | 111.3 | 1.5 | 166.9 | 224.5 | 1.5 | 336.7 | 152.1 | 1.5 | 228.2 | 116.3 | 1.5 | 177.5 |
| | Asc 10⁻⁵ | 103 | 1.5 | 154.4 | 140.6 | 1.5 | 211.1 | 135.5 | 1.5 | 203.3 | 109.7 | 1 | 109.7 |
| | Asc 10⁻⁷ | 55.9 | 1 | 55.9 | 89.6 | 1.5 | 134.4 | 48 | 1 | 48 | 43.5 | 1 | 43.5 |
| 93-30 | PBS | 73.3 | 1 | 73.3 | 69.2 | 1 | 69.2 | 51.1 | 1 | 51.1 | 65.1 | 1 | 65.1 |
| | His | 103 | 1.5 | 154.4 | 102.8 | 1.5 | 154.2 | 118.3 | 1.5 | 174.4 | 132.3 | 1.5 | 198.4 |
| | Asc 10⁻³ | 106.1 | 1.5 | 159.1 | 131.1 | 1.5 | 196.7 | 137.2 | 1.5 | 205.7 | 183.2 | 1.5 | 274.8 |
| | Asc 10⁻⁵ | 45.5 | 1.5 | 66.3 | 116.6 | 1.5 | 174.8 | 113.4 | 1.5 | 170 | 102.7 | 1.5 | 154.1 |
| | Asc 10⁻⁷ | 50.3 | 1 | 50.3 | 47.6 | 1 | 47.6 | 54 | 1 | 54 | 50.3 | 1 | 50.3 |
| 33-274 | PBS | 60.1 | 1 | 60.1 | 59.2 | 1 | 59.2 | 48.3 | 1 | 48.3 | 63.2 | 1 | 63.2 |
| | His | 115.5 | 1.5 | 173.3 | 102.6 | 1.5 | 163.9 | 112.2 | 1.6 | 168.3 | 135.7 | 1.5 | 203.6 |
| | Asc 10⁻³ | 110.3 | 1.5 | 165.4 | 173.6 | 1.5 | 260.4 | 191.3 | 1.5 | 286.9 | 171.3 | 1.5 | 256.9 |
| | Asc 10⁻⁵ | 60.5 | 1.5 | 90.7 | 120.8 | 1.5 | 181.1 | 115.5 | 1.5 | 173.3 | 95.5 | 1.5 | 143.2 |
| | Asc 10⁻⁷ | 34.5 | 1 | 34.5 | 90 | 1.5 | 135 | 89.2 | 1.5 | 133.9 | 64.6 | 1 | 64.6 |

Treatment Study

| | | | | | | | | | | | | | |
|---|---|---|---|---|---|---|---|---|---|---|---|---|---|
| 65-256 | PBS | 51.5 | 1 | 51.5 | 44.2 | 1 | 44.2 | 47.6 | 1 | 47.6 | 54.51 | 1 | 54.5 |
| | His | 169.9 | 1.5 | 254.9 | 106.8 | 1.5 | 159.8 | 117.2 | 1.5 | 175.7 | 114.7 | 1.5 | 172 |
| | Asc 10⁻³ | 145.2 | 1.5 | 217.8 | 135.9 | 1.5 | 203.8 | 111.4 | 1.5 | 167 | 133.2 | 1.5 | 199.9 |
| | Asc 10⁻⁵ | 112 | 1.5 | 168 | 111.6 | 1.5 | 167.3 | 81.7 | 1.5 | 122.6 | 100.6 | 1.5 | 150.9 |
| | Asc 10⁻⁷ | 74.1 | 1 | 74.1 | 72.2 | 1 | 72.2 | 45.4 | 1 | 45.4 | 97.9 | 1 | 97.9 |
| 65-271 | PBS | 72.2 | 1 | 72.2 | 37.6 | 1 | 37.6 | 67.2 | 1 | 67.2 | 45.8 | 1 | 45.8 |
| | His | 200.4 | 1.5 | 300.6 | 104.6 | 1.5 | 157 | 97.9 | 1.5 | 146.9 | 106 | 1.5 | 159 |
| | Asc 10⁻³ | 168 | 1.5 | 252 | 124.7 | 1.5 | 187.1 | 137.8 | 1.5 | 206.8 | 134.8 | 1.5 | 202.2 |
| | Asc 10⁻⁵ | 122.4 | 1 | 122.4 | 122 | 1.5 | 183 | 113.2 | 1.5 | 169.8 | 96 | 1.5 | 147 |
| | Asc 10⁻⁷ | 94.1 | 1 | 94.1 | 71.3 | 1.5 | 106.9 | 100.8 | 1.5 | 151.2 | 90.3 | 1.5 | 135.4 |
| 65-297 | PBS | 64 | 1 | 64 | 54.2 | 1 | 54.2 | 64 | 1 | 64 | 66.4 | 1 | 66.4 |
| | His | 53.9 | 1 | 53.9 | 89.1 | 1.5 | 133.7 | 141.5 | 1.5 | 212.4 | 103.8 | 1.5 | 155.8 |
| | Asc 10⁻³ | 116.5 | 1.5 | 174.7 | 149.6 | 1.5 | 224.4 | 115 | 1.5 | 172.5 | 148.8 | 1.5 | 223.3 |
| | Asc 10⁻⁵ | 77.7 | 1.5 | 116.6 | 87.5 | 1.5 | 131.2 | 109.8 | 1.5 | 184.7 | 117.8 | 1.5 | 176.7 |
| | Asc 10⁻⁷ | 57.4 | 1 | 57.4 | 62.2 | 1 | 52.2 | 61.5 | 1 | 61.5 | 99.8 | 1.5 | 149.5 |
| 65-304 | PBS | 56.3 | 1 | 56.3 | 51.8 | 1 | 51.8 | 36 | 1 | 36 | 57.6 | 1 | 57.6 |
| | His | 48.4 | 1 | 48.4 | 132.7 | 1.5 | 199 | 131 | 1.5 | 196.5 | 100.3 | 1.5 | 150.5 |
| | Asc 10⁻³ | 134.4 | 1.5 | 201.6 | 138 | 1.5 | 207 | 126.4 | 1.5 | 189.6 | 136.6 | 1.5 | 204.9 |
| | Asc 10⁻⁵ | 100.8 | 1.5 | 151.2 | 72.2 | 1.5 | 108.3 | 116.8 | 1.5 | 176.2 | 126.7 | 1.5 | 190.1 |
| | Asc 10⁻⁷ | 57.3 | 1 | 57.3 | 49 | 1 | 49 | 53.3 | 1 | 53.3 | 95.2 | 1.5 | 142.8 |

Individual animal data (weeks 8-14)

| | Week 8 | | | Week 10 | | | Week 12 | | | Week 14 | | |
|---|---|---|---|---|---|---|---|---|---|---|---|---|
| | Wheal | Clinical | | Wheal | Clinical | | Wheal | Clinical | | Wheal | Clinical | |
| Animal # | (mm²) | Score | Total | (mm²) | Score | Total | (mm²) | Score | Total | (mm²) | Score | Total |

Control Study

| | | | | | | | | | | | |
|---|---|---|---|---|---|---|---|---|---|---|---|
| 93-282 | PBS | 50.6 | 1 | 50.6 | 52 | 1 | 52 | 67.5 | 1 | 67.5 | NOT |
| | His | 106.6 | 1.5 | 159.8 | 111.3 | 1.5 | 167 | 130 | 1.5 | 195 | DONE |
| | Asc 10⁻³ | 128.6 | 1.5 | 192.9 | 126.7 | 1.5 | 190.1 | 142.4 | 1.5 | 213.6 | |
| | Asc 10⁻⁵ | 86.7 | 1.5 | 130.1 | 118.6 | 1.5 | 177.9 | 89 | 1.5 | 133.5 | |
| | Asc 10⁻⁷ | 54.4 | 1 | 54.4 | 49 | 1 | 49 | 50.7 | 1 | 50.7 | |
| 43-53 | PBS | 84.6 | 1 | 84.6 | 58.5 | 1 | 58.5 | 60 | 1 | 60 | NOT |
| | His | 131.6 | 1.5 | 197.4 | 122 | 1.5 | 183.1 | 153.8 | 1.5 | 230.6 | DONE |
| | Asc 10⁻³ | 124.3 | 1.5 | 1 [symbol] .4 | 142.7 | 1.5 | 214.1 | 140 | 1.5 | 210 | |
| | Asc 10⁻⁵ | 10 [symbol] .8 | 1.5 | 163.2 | 133.1 | 1.5 | 200 | 111.3 | 1.5 | 167 | |
| | Asc 10⁻⁷ | 80.3 | 1 | 80.3 | 58.1 | 1 | 58.1 | 70.3 | 1 | 70.3 | |
| 93-30 | PBS | 70.5 | 1 | 70.5 | 49 | 1 | 49 | 67.2 | 1 | 67.2 | NOT |
| | His | 124.3 | 1.5 | 186.5 | 123.8 | 1.5 | 185.8 | 128.3 | 1.5 | 192.4 | DONE |
| | Asc 10⁻³ | 125.4 | 1.5 | 188.2 | 135.7 | 1.5 | 203.6 | 139.2 | 1.5 | 208.8 | |
| | Asc 10⁻⁵ | 114.1 | 1.5 | 171.2 | 113.4 | 1.5 | 170.1 | 123.2 | 1.5 | 184.8 | |
| | Asc 10⁻⁷ | 62.1 | 1 | 62.1 | 54.6 | 1 | 54.6 | 78.7 | 1 | 78.7 | |
| 33-274 | PBS | 60.8 | 1 | 60.8 | 60.1 | 1 | 60.1 | 41.6 | 1 | 41.5 | NOT |
| | His | 117.3 | 1.5 | 176 | 102.9 | 1.5 | 154.4 | 106 | 1.5 | 159 | DONE |
| | Asc 10⁻³ | 139.2 | 1.5 | 208.9 | 151.3 | 1.5 | 227 | 126.5 | 1.5 | 189.8 | |
| | Asc 10⁻⁵ | 115. [symbol] | 1.5 | 173.7 | 96.3 | 1.5 | 144.5 | 102.7 | 1.5 | 154.1 | |
| | Asc 10⁻⁷ | 69.7 | 1.5 | 104.6 | 56.9 | 1.5 | 85.3 | 94.8 | 1.5 | 142.1 | |

Treatment Study

| | | | | | | | | | | | | | |
|---|---|---|---|---|---|---|---|---|---|---|---|---|---|
| 65-256 | PBS | 50.7 | 1 | 50.7 | 56.2 | 1 | 56.2 | 63.2 | 1 | 63.2 | 67.2 | 1 | 67.2 |
| | His | 119.5 | 1.5 | 179.2 | 96.6 | 1.5 | 144.9 | 125.4 | 1.5 | 188.2 | 108.9 | 1.5 | 163.4 |
| | Asc 10⁻³ | 122.9 | 1.5 | 184.3 | 83.7 | 1.5 | 125.5 | 163 | 1.5 | 244.5 | 161.2 | 1.5 | 242.8 |
| | Asc 10⁻⁵ | [symbol] 8 | 1.5 | 147 | 55.6 | 1 | 55.6 | 107 | 1.5 | 160.5 | 131.6 | 1.5 | 197.4 |
| | Asc 10⁻⁷ | 45.8 | 1 | 45.8 | 34.8 | 1 | 34.8 | 51.1 | 1 | 51.1 | 58 | 1 | 58 |
| 65-271 | PBS | 57 | 1 | 57 | 36.6 | 1 | 36.6 | 42.2 | 1 | 42.2 | 50.9 | 1 | 50.9 |
| | His | 114.5 | 1.5 | 171.7 | 121.4 | 1.5 | 182.1 | 108.1 | 1.5 | 162.2 | 105.3 | 1.5 | 157.9 |
| | Asc 10⁻³ | 91.2 | 1.5 | 136.8 | 90.5 | 1.5 | 135.7 | 108.8 | 1.5 | 163.2 | 118.1 | 1.5 | 177.1 |
| | Asc 10⁻⁵ | 61.6 | 1.5 | 92.4 | 55.6 | 1 | 55.6 | 81.7 | 1.5 | 122.6 | 86.3 | 1.5 | 129.5 |
| | Asc 10⁻⁷ | 42.9 | 1 | 42.9 | 35 | 1 | 35 | 44.1 | 1 | 44.1 | 43.86 | 1 | 43.86 |
| 65-297 | PBS | 60 | 1 | 60 | 51.6 | 1 | 51.6 | 56.2 | 1 | 56.2 | 60.7 | 1 | 60.7 |
| | His | 109.5 | 1.5 | 164.2 | 53.5 | 1.5 | 80.2 | 115.6 | 1.5 | 173.5 | 121 | 1.5 | 181.5 |
| | Asc 10⁻³ | 109.8 | 1.5 | 164. [symbol] | 108.2 | 1.5 | 162.2 | 153.4 | 1.5 | 230.1 | 132.1 | 1.5 | 198.1 |
| | Asc 10⁻⁵ | 54.2 | 1 | 54.2 | 54.3 | 1 | 54.3 | 97.01 | 1.5 | 145.5 | 126.3 | 1.5 | 189.4 |
| | Asc 10⁻⁷ | 37.2 | 1 | 37.2 | 41.8 | 1 | 41.8 | 50.7 | 1 | 50.7 | 62.7 | 1 | 62.7 |
| 65-304 | PBS | 47.6 | 1 | 47.6 | 39.7 | 1 | 39.7 | 67.9 | 1 | 67.9 | 60.84 | 1 | 60.84 |
| | His | 142.4 | 1.5 | 213.6 | 122.1 | 1.5 | 183.1 | 127.7 | 1.5 | 191.5 | 138.6 | 1.5 | 207.9 |
| | Asc 10⁻³ | 126.1 | 1.5 | 159.2 | 85.4 | 1.5 | 128.2 | 150.9 | 1.5 | 226.4 | 11 [symbol] .3 | 1.5 | 174.4 |
| | Asc 10⁻⁵ | 68 | 1 | 68 | 40.1 | 1 | 40.1 | 103.7 | 1.5 | 155.6 | 109.8 | 1.6 | 164.6 |
| | Asc 10⁻⁷ | 52 | 1 | 52 | 35.4 | 1 | 35.4 | 54 | 1 | 54 | 54.3 | 1 | 54.3 |

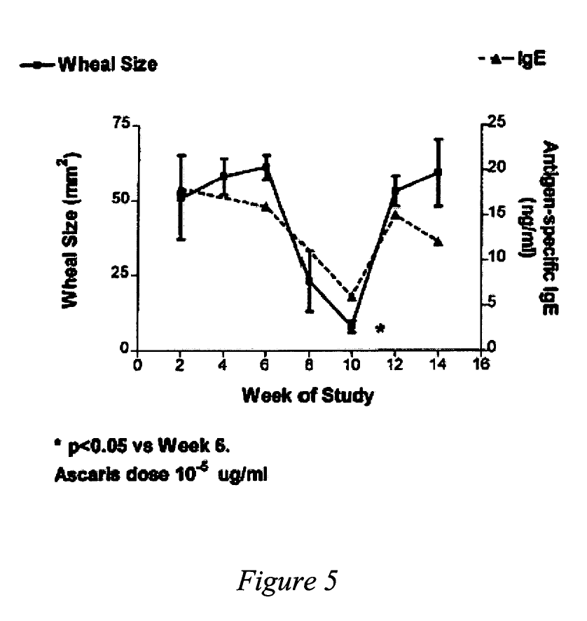

Analysis of plasma specific-IgE levels, using an ELISA method, demonstrated a four-fold reduction that is similar to the reduction observed in the cutaneous wheal response (see FIG. 5). MhIL-4 is a recombinant human protein, so multiple administrations of the drug induced an immunogenic response in the monkeys. Binding of mhIL-4 to its receptor was monitored using plasmon surface resonance (see FIG. 6). FIG. 6 shows that the pronounced reduction in plasmon resonance units occurred at week 10, suggesting an inhibition of the binding of mhIL-4 to the IL-4 receptor from that time onward. These studies also suggest that the loss of activity of mhIL-4, observed in both the wheal responses and the levels of plasma IgE, may be a result of the production of neutralizing antibodies.

The results of this study demonstrate that repeated administration of the mhIL-4 significantly reduces circulating IgE levels and inhibits antigen-induced cutaneous wheal and flare responses in cynomolgus monkeys. Similar and more profound results are expected for the human clinical trials.

Although the present process has been described with reference to specific details of certain embodiments thereof in the above example, it will be understood that modifications and variations are encompassed within the spirit and scope of the invention. Accordingly, the invention is limited only by the following claims.

We claim:

1. A method for suppressing or inhibiting a dermatitis response in a subject comprising:
administering to a subject in need thereof a therapeutic effective amount of a mutant human IL-4 protein, or functional fragment thereof, wherein the protein comprises at least a first modification of replacing one or more of the amino acids occurring in the wild-type human IL-4 protein at positions 121, 124 and/or 125 with another natural amino acid.

2. The method of claim 1, wherein the therapeutic effective amount is from about 0.3 mg/kg to about 0.6 mg/kg daily.

3. The method of claim 1, further comprising a second modification selected from a group consisting of a modification at the N-terminus, C-terminus, deletion of potential glycosylation sites therein, and/or coupling of the protein to a non-protein polymer.

4. The method of claim 3, wherein the modification of the N-terminus is deletion or insertion of one or more amino acids.

5. The method of claim 3, wherein the modification of the C-terminus is deletion or insertion of one or more amino acids.

6. The method of claim 3, wherein the non-protein polymer is selected from the group consisting of polyethylene glycol (PEG), polypropylene glycol and polyoxyalkylenes.

7. The method of claim 6, wherein the human IL-4 mutein is coupled to the non-protein polymer at amino acid residue position 38, 102 and/or 104.

8. The method of claim 6, wherein the human IL-4 mutein is coupled to the non-protein polymer at amino acid residue position 38 of IL-4.

9. The method of claim 6, wherein the human IL-4 mutein is coupled to the non-protein polymer at amino acid residue position 102.

10. The method of claim 6, wherein the human IL-4 mutein is coupled to the non-protein polymer at amino acid residue position 104.

11. The method of claims 6-10 wherein the non-protein polymer is polyethylene glycol PEG.

12. The method of claim 1, wherein the human IL-4 mutein modification comprises substitutions R121D or R121E.

13. The method of claim 12, wherein the human IL-4 mutein modification comprises substitutions R121D.

14. The method of claim 1, wherein the human IL-4 mutein modification comprises substitutions Y124D or Y124E.

15. The method of claim 14, wherein the human IL-4 mutein modification comprises substitutions Y124D.

16. The method of claim 1, wherein the human IL-4 mutein modification comprises substitutions S125D or S125E.

17. The method of claim 16, wherein the human IL-4 mutein modification comprises substitutions S125D.

18. The method of claim 1, wherein the human IL-4 mutein modification comprises substitutions R121D and Y124D.

19. The method of claim 1, wherein the human IL-4 mutein modification comprises substitutions R121D, Y124D and S125D.

20. The method of claim 3, wherein the modification of the N-terminus is insertion of an amino acid residue at position +2.

21. The method of claim 1, wherein the dermatitis is an allergic or atopic reaction.

22. The method of claim 1, wherein the dermatitis is a hypersensitivity reaction.

23. The method of claim 22, wherein the hypersensitivity reaction is contact dermatitis.

24. The method of claim 22, wherein the hypersensitivity reaction is atopic dermatitis.

25. The method of claim 1, wherein the subject is human.

26. A method for treating a subject having atopic dermatitis, comprising,
administering to the subject having atopic dermatitis a therapeutically effective amount of a composition comprising a mutant human IL-4 protein, wherein the protein comprises a first modification of replacing one or more of the amino acids occurring in the wild-type human IL-4 protein at positions 121, 124 and/or 125 with another natural amino acid, and a second modification selected from a group consisting of a modification at the N-terminus, C-terminus, deletion of potential glycosylation sites therein, and/or coupling of the protein to a non-protein polymer, and wherein the therapeutic effective amount is from about 0.3 mg/kg to about 0.6 mg/kg daily.

27. The method of claim 26, wherein the subject is human.

28. The method of claim 26, wherein the non-protein polymer is selected from the group consisting of polyethylene glycol (PEG), polypropylene glycol and polyoxyalkylenes.

29. The method of claim 26, wherein the human IL-4 mutein is coupled to the non-protein polymer at amino acid residue position 38, 102 and/or 104.

30. The method of claim 29, wherein the human IL-4 mutein is coupled to the non-protein polymer at amino acid residue position 38.

31. The method of claim 29, wherein the human IL-4 mutein is coupled to the non-protein polymer at amino acid residue position 102.

32. The method of claim 29, wherein the human IL-4 mutein is coupled to the non-protein polymer at amino acid residue position 104.

33. The method of claim 30-32, wherein the non-protein polymer is polyethylene glycol PEG.

34. The method of claim 26, wherein the human IL-4 mutein modification comprises substitutions R121D or R121E.

35. The method of claim 34, wherein the human IL-4 mutein modification comprises substitutions R121D.

36. The method of claim 26, wherein the human IL-4 mutein modification comprises substitutions Y124D or Y124E.

37. The method of claim 36, wherein the human IL-4 mutein modification comprises substitutions Y124D.

38. The method of claim 26, wherein the human IL-4 mutein modification comprises substitutions S125D or S125E.

39. The method of claim 38, wherein the human IL-4 mutein modification comprises substitutions S125D.

40. The method of claim 26, wherein the human IL-4 mutein modification comprises substitutions R121D and Y124D.

41. The method of claim 26, wherein the human IL-4 mutein modification comprises substitutions R121D, Y124D and S125D.

42. The method of claim 26, wherein the modification of the N-terminus is deletion or insertion of one or more amino acids.

43. The method of claim 42, wherein the modification of the N-terminus is insertion of an amino acid residue at position +2.

44. The method of claim 26, wherein the modification of the C-terminus is deletion or insertion of one or more amino acids.
